SEQUENCE LISTING (32 sequences)

SEQ ID NO: 1 - DNA, 1416 bp, Bacillus subtilis
  misc_feature: YwfE (wild type)
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcataaaagataaagactattttaagagtttagctgattttgagcatcctgattccatttattgggcgcatgaggatcataacaagcctgaggaagaggtcgtcgagcaaatcgtcaaggttgccgaaatgtttggggcggatgccatcacaacaaacaatgaattattcattgctccgatggcgaaagcctgtgaacgtctgggcctgagaggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttcaatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaagggcttggcctgcaaaattgcgcaacacatacagagatcaagctaatgaaaaacagagaaccgggtttaatagagtcggcagccagattcgcaggctggaatatgattcctaatattaaaaaggtctttggccttgatatggcgcaattattattagatgtcctctgtttcggaaaagacgccgatctgccggacggattattggatcaagagccttattatgttgccgactgccatttgtacccgcagcatttcaaacaaaatggccagattccagaaaccgctgaggatttggtcattgaagcgatcgatattccggacgggcttttaaaaggggatactgaaatcgtttcattttcagccgcagcaccaggcacttcagttgatttgacattgtttgaagctttcaattccattgctgcatttgaactgaaaggcagtaattcacaggatgtggctgaatcaatcagacaaattcagcagcatgcaaagctgacggcaaagtatgtgctgccagta

SEQ ID NO: 2 - DNA, 33 bp, Artificial Sequence
  N108E_sense
acaaacgaagaattattcattgctccgatggcg

SEQ ID NO: 3 - DNA, 33 bp, Artificial Sequence
  N108E_antisense
taattcttcgtttgttgtgatggcatccgcccc

SEQ ID NO: 4 - DNA, 33 bp, Artificial Sequence
  I112V_sense
ttattcgtggctccgatggcgaaagcctgtgaa

SEQ ID NO: 5 - DNA, 33 bp, Artificial Sequence
  I112V_antisense
cggagccacgaataattcattgtttgttgtgat

SEQ ID NO: 6 - DNA, 1419 bp, Bacillus subtilis
  misc_feature: YwfE (mutant type N108E)
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcataaaagataaagactattttaagagtttagctgattttgaacaccctgattccatttattgggcgcatgaagatcataacaagcctgaggaagaggtcgtcgagcaaatcgtcaaggttgccgaaatgtttggggcggatgccatcacaacaaacgaagaattattcattgctccgatggcgaaagcctgtgaacgtctgggcttgagaggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttctatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaagggcttggactgcaaaattgcgcaacacatacagagatcaagctaatgaaaaacagagaaccgggtttaatagagtcggcagccagatttgccggctggaatatgatccccaatattaaaaaggtctttggccttgatatggcgcaattattattagatgtcctctgtttcggaaaagacgccgatctgccggacggattattggatcaagagccttattatgttgccgactgccatttgtacccgcagcatttcaaacaaaatggccaaattcctgaaactgctgaggatttggtcattgaagcgatcgatattccggacgggcttttaaaaggggatactgaaatcgtttctttttcggccgcagcaccaggcacttcagttgatttgacattgtttgaagctttcaattccattgctgcatttgaactgaaaggcagtaattcacaggatgtggctgaatcaatcagacaaattcagcagcatgcgaagctgacggcaaagtatgtgctgccagtatga

SEQ ID NO: 7 - DNA, 1416 bp, Bacillus subtilis
  misc_feature: YwfE (mutant type I112V)
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcataaaagataaagactattttaagagtttagctgattttgagcatcctgattccatttattgggcgcatgaggatcataacaagcctgaggaagaggtcgtcgagcaaatcgtcaaggttgccgaaatgtttggggcggatgccatcacaacaaacaatgaattattcgttgctccgatggcgaaagcctgtgaacgtctgggcctgagaggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttcaatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaagggcttggcctgcaaaattgcgcaacacatacagagatcaagctaatgaaaaacagagaaccgggtttaatagagtcggcagccagattcgcaggctggaatatgattcctaatattaaaaaggtctttggccttgatatggcgcaattattattagatgtcctctgtttcggaaaagacgccgatctgccggacggattattggatcaagagccttattatgttgccgactgccatttgtacccgcagcatttcaaacaaaatggccagattccagaaaccgctgaggatttggtcattgaagcgatcgatattccggacgggcttttaaaaggggatactgaaatcgtttcattttcagccgcagcaccaggcacttcagttgatttgacattgtttgaagctttcaattccattgctgcatttgaactgaaaggcagtaattcacaggatgtggctgaatcaatcagacaaattcagcagcatgcaaagctgacggcaaagtatgtgctgccagta

SEQ ID NO: 8 - DNA, 1419 bp, Bacillus subtilis
  misc_feature: YwfE (mutant type N108E/H378K)
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcataaaagataaagactattttaagagtttagctgattttgaacaccctgattccatttattgggcgcatgaagatcataacaagcctgaggaagaggtcgtcgagcaaatcgtcaaggttgccgaaatgtttggggcggatgccatcacaacaaacgaagaattattcattgctccgatggcgaaagcctgtgaacgtctgggcttgagaggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttctatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaagggcttggactgcaaaattgcgcaacacatacagagatcaagctaatgaaaaacagagaaccgggtttaatagagtcggcagccagatttgccggctggaatatgatccccaatattaaaaaggtctttggccttgatatggcgcaattattattagatgtcctctgtttcggaaaagacgccgatctgccggacggattattggatcaagagccttattatgttgccgactgcaaattgtacccgcagcatttcaaacaaaatggccaaattcctgaaactgctgaggatttggtcattgaagcgatcgatattccggacgggcttttaaaaggggatactgaaatcgtttctttttcggccgcagcaccaggcacttcagttgatttgacattgtttgaagctttcaattccattgctgcatttgaactgaaaggcagtaattcacaggatgtggctgaatcaatcagacaaattcagcagcatgcgaagctgacggcaaagtatgtgctgccagtatga

SEQ ID NO: 9 - DNA, 1416 bp, Bacillus subtilis
  misc_feature: YwfE (mutant type I112V/H378K)
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcataaaagataaagactattttaagagtttagctgattttgagcatcctgattccatttattgggcgcatgaggatcataacaagcctgaggaagaggtcgtcgagcaaatcgtcaaggttgccgaaatgtttggggcggatgccatcacaacaaacaatgaattattcgttgctccgatggcgaaagcctgtgaacgtctgggcctgagaggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttcaatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaagggcttggcctgcaaaattgcgcaacacatacagagatcaagctaatgaaaaacagagaaccgggtttaatagagtcggcagccagattcgcaggctggaatatgattcctaatattaaaaaggtctttggccttgatatggcgcaattattattagatgtcctctgtttcggaaaagacgccgatctgccggacggattattggatcaagagccttattatgttgccgactgcaaattgtacccgcagcatttcaaacaaaatggccagattccagaaaccgctgaggatttggtcattgaagcgatcgatattccggacgggcttttaaaaggggatactgaaatcgtttcattttcagccgcagcaccaggcacttcagttgatttgacattgtttgaagctttcaattccattgctgcatttgaactgaaaggcagtaattcacaggatgtggctgaatcaatcagacaaattcagcagcatgcaaagctgacggcaaagtatgtgctgccagta

SEQ ID NO: 10 - DNA, 33 bp, Artificial Sequence
  H378K_sense
gactgcaaattgtacccgcagcatttcaaacaa

SEQ ID NO: 11 - DNA, 33 bp, Artificial Sequence
  H378K_antisense
gtacaatttgcagtcggcaacataataaggctc

SEQ ID NO: 12 - protein, 472 aa, Bacillus subtilis
  MISC_FEATURE: YwfE (wild type)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNNELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 13 - protein, 472 aa, Bacillus subtilis
  MISC_FEATURE: YwfE (mutant type N108A)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNAELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 14 - protein, 472 aa, Bacillus subtilis
  MISC_FEATURE: YwfE (mutant type N108E)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNEELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 15 - protein, 472 aa, Bacillus subtilis
  MISC_FEATURE: YwfE (mutant type N108Q)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNQELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 16 - protein, 472 aa, Bacillus subtilis
  MISC_FEATURE: YwfE (mutant type I112V)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNNELFVAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 17 - protein, 472 aa, Bacillus subtilis
  MISC_FEATURE: YwfE (mutant type H378K)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNNELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCKLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 18 - protein, 472 aa, Bacillus subtilis
  MISC_FEATURE: YwfE (mutant type H378R)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNNELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCRLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 19 - protein, 472 aa, Bacillus subtilis
  MISC_FEATURE: YwfE (mutant type N108A/I112V)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNAELFVAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 20 - protein, 472 aa, Bacillus subtilis
  MISC_FEATURE: YwfE (mutant type N108A/H378K)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNAELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCKLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 21 - protein, 472 aa, Bacillus subtilis
  MISC_FEATURE: YwfE (mutant type N108A/H378R)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNAELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCRLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 22 - protein, 472 aa, Bacillus subtilis
  MISC_FEATURE: YwfE (mutant type N108E/I112V)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNEELFVAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 23 - protein, 472 aa, Bacillus subtilis
  MISC_FEATURE: YwfE (mutant type N108E/H378K)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNEELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCKLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 24 - protein, 472 aa, Bacillus subtilis
  MISC_FEATURE: YwfE (mutant type N108E/H378R)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNEELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCRLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 25 - protein, 472 aa, Bacillus subtilis
  MISC_FEATURE: YwfE (mutant type N108Q/I112V)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNQELFVAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 26 - protein, 472 aa, Bacillus subtilis
  MISC_FEATURE: YwfE (mutant type N108Q/H378K)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNQELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCKLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 27 - protein, 472 aa, Bacillus subtilis
  MISC_FEATURE: YwfE (mutant type N108Q/H378R)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNQELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCRLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 28 - protein, 472 aa, Bacillus subtilis
  MISC_FEATURE: YwfE (mutant type I112V/H378K)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNNELFVAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCKLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 29 - protein, 472 aa, Bacillus subtilis
  MISC_FEATURE: YwfE (mutant type I112V/H378R)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNNELFVAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCRLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 30 - protein, 472 aa, Bacillus subtilis
  MISC_FEATURE: YwfE (mutant type N108A/I112V/H378R)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNAELFVAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCRLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 31 - protein, 472 aa, Bacillus subtilis
  MISC_FEATURE: YwfE (mutant type N108Q/I112V/H378K)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNQELFVAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCKLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 32 - protein, 472 aa, Bacillus subtilis
  MISC_FEATURE: YwfE (mutant type N108Q/I112V/H378R)
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNQELFVAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCRLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

(19) United States
(12) Patent Application Publication
(10) Pub. No.: US 2020/0263222 A1
(43) Pub. Date: Aug. 20, 2020

(54) PRODUCTION METHOD OF IMIDAZOLEDIPEPTIDE

(71) Applicant: TOKAI BUSSAN CO., LTD., Tokyo (JP)

(72) Inventor: Kuniki KINO, Tokyo (JP)

(21) Appl. No.: 16/789,202

(22) Filed: Feb. 12, 2020

(30) Foreign Application Priority Data
Feb. 13, 2019 (JP) 2019-023453

(51) Int. Cl. C12P 21/02; C07K 5/078

(52) U.S. Cl. CPC C12P 21/02; C12Y 603/02028; C12Y 201/01022; C07K 5/06147

(57) ABSTRACT

A method for producing imidazole dipeptide using a microorganism having imidazole dipeptide synthesis activity, the production method not including adding ATP, or including adding an amount of ATP that is less than the amount of imidazole dipeptide that is produced in terms of the number of moles, by utilizing an ATP supply system of the microorganism.

### CROSS-REFERENCE TO RELATED APPLICATION

This application claims priority to Japanese Patent Application No. 2019-023453, filed Feb. 13, 2019, the content of which is hereby incorporated herein by reference.

### BACKGROUND

### Field of the Invention

The present invention relates to a method for producing imidazole dipeptides, such as carnosine, anserine, and balenine.

### Background Information

Imidazole dipeptide is a generic term for peptides to which an amino-acid residue containing an imidazole group is bonded, and includes dipeptides containing a β-alanine and a histidine residue or a derivative thereof, such as carnosine (carnosine: β-alanyl-L-histidine), anserine (anserine: β-alanyl-3-methyl-L-histidine), and balenine (balenine: Nα-β-alanyl-1-methyl-L-histidine). These dipeptides are contained in abundance in the breast meat of birds that fly long distances as well as the muscles of marine animals that swim long distances, such as tuna, bonito, and whales, and are known to have anti-fatigue effects. That is, the ability to scavenge active oxygen and lower blood pressure, anti-inflammatory effects, uric acid level lowering effects, etc., These imidazole dipeptides can be extracted from the muscle of domestic animals such as chickens are utilized as food supplements (See Song B. et al, Nutr. Res. Pract. 2014, 8: 3-10, Bellia F. et al., Molecules 2014, 19: 2299-2329 and Boldyrev A. A. et al., Physiol. Rev. 2013, 93: 1803-45).

Attempts have been made to produce a mutant of L-amino acid α-ligase and apply it to carnosine synthesis, but this is not always sufficient in terms of efficiency and cost (See Japanese Laid-Open Patent Application No. 2013-081405).

### SUMMARY

With the object to establish an easy and low-cost method for producing imidazole dipeptide, the present inventors prepared various YwfE mutants derived from Bacillus subtilis, which is an L-amino acid α-ligase (Japanese Laid Open Patent Application No. 2018-102287). However, since L-amino acid α-ligase couples with hydrolysis reaction of ATP at the time of condensation of β-alanine and histidine or a derivative thereof, in the prior art, it is necessary to add ATP at the time of production reaction of imidazole dipeptide.

On the other hand, an enzyme that converts carnosine to anserine is known from the prior art (See Drozak J. et al., J. Biol. Chem. 2015, 290: 17190-17205). However, a method for producing anserine from carnosine using Escherichia coli (E. coli) is not known.

It has been determined that a highly efficient and low-cost method for producing carnosine, anserine, and balenine has not yet been established. Therefore, an object of the present invention is to provide a production method that is capable of producing an imidazole peptide, selected from carnosine, anserine, and balenine, in a highly efficient and low-cost manner.

It was observed that imidazole dipeptide can be synthesized by coupling the hydrolysis reaction of ATP disclosed in Japanese Laid Open Patent Application No. 2018-102287 and forming a peptide bond using unprotected amino acid as a substrate, as an enzymatic synthesis method of imidazole dipeptide. Moreover, imidazole dipeptide can be produced highly efficiently and at a low cost by combining an ATP supply system with a microorganism having an imidazole dipeptide synthesis activity.

Furthermore, a method for producing anserine from carnosine has been established.

Specifically, embodiments of the present invention described herein provide a method for producing imidazole dipeptide using a microorganism having imidazole dipeptide synthesis activity, wherein the production method does not comprise adding ATP, or comprises adding an amount of ATP that is less than the amount of imidazole dipeptide that is produced in terms of number of moles, by utilizing the ATP supply system of the microorganism.

In one embodiment of a method for producing imidazole dipeptide according to the present invention, the imidazole dipeptide can be selected from carnosine, anserine, and balenine.

In one embodiment of a method for producing imidazole dipeptide according to the present invention, the microorganism can be a microorganism expressing L-amino acid α-ligase.

In one embodiment of a method for producing imidazole dipeptide according to the present invention, the L-amino acid α-ligase can be a protein having imidazole dipeptide synthesis activity.

In one embodiment of a method for producing imidazole dipeptide according to the present invention, the L-amino acid α-ligase is a mutant L-amino acid α-ligase in which are substituted 1 to 3 amino-acid residues of a wild-type YwfE amino-acid sequence that has an amino-acid sequence represented by SEQ ID NO: 12, and can include a substitution of an amino-acid residue corresponding to the 108th asparagine (N) residue from the N-terminus of SEQ ID NO: 12 with an amino acid selected from alanine (A), glutamic acid (E), and glutamine (Q), a substitution of an amino-acid residue corresponding to the 112th isoleucine (I) residue from the N-terminus of SEQ ID NO: 12 with valine (V), and/or a substitution of an amino-acid residue corresponding to the 378th histidine (H) residue from the N-terminus of SEQ ID NO: 12 with an amino acid selected from lysine (K) or arginine (R).

In one embodiment of a method for producing imidazole dipeptide according to the present invention, the production method can include a bacterial reaction method or a fermentation method.

One embodiment of a method for producing imidazole dipeptide according to the present invention can comprise adding a glucide with respect to which the microorganism has a metabolic ability.

In one embodiment of a method for producing imidazole dipeptide according to the present invention, the microorganism can be a peptidase-deficient strain.

In one embodiment of a method for producing imidazole dipeptide according to the present invention, the peptidase can be PepD.

In addition, embodiments of the present invention provide a method for producing anserine in which the imidazole dipeptide is carnosine, comprising further reacting carnosine produced by the microorganism with an enzyme having carnosine N-methyltransferase activity.

In one embodiment of a method for producing anserine according to the present invention, a microorganism expressing an enzyme having carnosine N-methyltransferase activity can be used.

In one embodiment of a method for producing anserine according to the present invention, a microorganism coexpressing an enzyme having carnosine synthesis activity and an enzyme having carnosine N-methyltransferase activity can be used as the L-amino acid α-ligase.

In one embodiment of a method for producing anserine according to the present invention, the enzyme having the carnosine N-methyltransferase activity can be expressed by SEQ ID NO: 36.

### BRIEF DESCRIPTION OF THE DRAWINGS

The invention will be explained in more detail hereinafter with reference to the drawings.

FIG. 1 is a view showing a result of evaluating the effect of peptidase in carnosine degradation. “Wild strain” indicates the use of a wild strain, and “ΔPepD” indicates the use of a PedD-deficient strain.

FIG. 2 is a view showing the results of evaluating the effect of adding glucose in carnosine synthesis by means of a bacterial reaction method. “WT/NEHK” indicates a transformant expressing a double-mutant YwfE (N108E/H378K) using a wild strain as a host. “G+” indicates a glucose-added group and “G−” a glucose-free group.

FIG. 3 is a view showing the results of evaluating the effect of adding glucose in anserine synthesis by means of a bacterial reaction method. “WT/IVHK” indicates a transformant expressing a double-mutant YwfE (I112V/H378K) using a wild strain as a host, and “ΔPepD/IVHK” indicates a transformant expressing a double-mutant YwfE (I112V/H378K) using a PepD-deficient strain as the host. “G+” indicates a glucose-added group and “G−” a glucose-free group.

FIG. 4 is an HPLC chromatogram showing an evaluation of anserine synthesis in a bacterial reaction method using a transformant expressing carnosine N-methyltransferase (YNL092W).

### DETAILED DESCRIPTION OF THE EMBODIMENTS

Method for Producing Imidazole Dipeptide

One embodiment of the present invention is a method for producing imidazole dipeptide using a microorganism having imidazole dipeptide synthesis activity.

More specifically, one embodiment of the present invention is a method for producing imidazole dipeptide using a microorganism having an imidazole dipeptide synthesis activity, wherein the production method does not comprise adding ATP, or comprises adding an amount of ATP that is less than the amount of imidazole dipeptide that is produced in terms of the number of moles, by utilizing the ATP supply system of the microorganism.

As described herein, a protein sequence is described by a notational convention well-known to those skilled in the art, using one or three letters of an amino acid. As described herein, the amino acid is the L-form unless otherwise specified. In addition, when a mutant protein is represented, a one-letter notation of the amino acid in which the mutation of the wild-type protein is introduced, a number representing the mutation position, and a one-letter notation of the mutated amino acid are used, which is method well-known to those skilled in the art.

In the production method according to one embodiment of the present invention, an example of the microorganism can include a microorganism expressing L-amino acid α-ligase.

As described herein, the L-amino acid α-ligase is not necessarily limited as long as it is an enzyme having an imidazole dipeptide synthesis activity, and an example of the L-amino acid α-ligase is a mutant L-amino acid α-ligase containing a substitution of some of the amino-acid sequence of a wild-type YwfE having an amino-acid sequence represented by SEQ ID NO: 12.

More specifically, the L-amino acid α-ligase can be a mutant L-amino acid α-ligase in which are substituted 1 to 3 amino-acid residues of a wild-type YwfE amino-acid sequence that has an amino-acid sequence represented by SEQ ID NO: 12, and preferably is a protein including

a substitution of an amino-acid residue corresponding to the 108th asparagine (N) residue from the N-terminus of SEQ ID NO: 12 with an amino acid selected from alanine (A), glutamic acid (E), and glutamine (Q),

a substitution of an amino-acid residue corresponding to the 112th isoleucine (I) residue from the N-terminus of SEQ ID NO: 12 with valine (V),

and/or

a substitution of an amino-acid residue corresponding to the 378th histidine (H) residue from the N-terminus of SEQ ID NO: 12 with an amino acid selected from lysine (K) or arginine (R).

More preferable examples of the protein can include:

(i) a protein having an amino-acid sequence of SEQ ID NOs: 13 to 32,
(ii) a protein having a homology of 80% or more, preferably 85% or more, more preferably 90% or more, and most preferably 95% or more with a protein having an amino-acid sequence of SEQ ID NOs: 13 to 32, and that has L-amino acid α-ligase activity, and
(iii) a protein comprising an amino-acid sequence in which one or a plurality of amino-acid residues of an amino-acid sequence of SEQ ID NOs: 13 to 32 are deleted, substituted, inserted, and/or added, and that has L-amino acid α-ligase activity.

The mutant L-amino acid α-ligase can be produced and used according to the method disclosed in Japanese Laid Open Patent Application No. 2018 and Drozak J. et al., J. Biol. Chem. 2015, 290: 17190-17205.

As described herein, examples of the L-amino acid α-ligase activity refer to imidazole dipeptide synthesizing activity, and examples of imidazole dipeptide produced by the L-amino acid α-ligase activity include carnosine, anserine, and balenine.

That is, preferred examples of the L-amino acid α-ligase activity can include carnosine synthesizing activity represented by the following formula 1, anserine synthesizing activity represented by the following formula 2, and/or balenine synthesizing activity represented by the following formula 3.

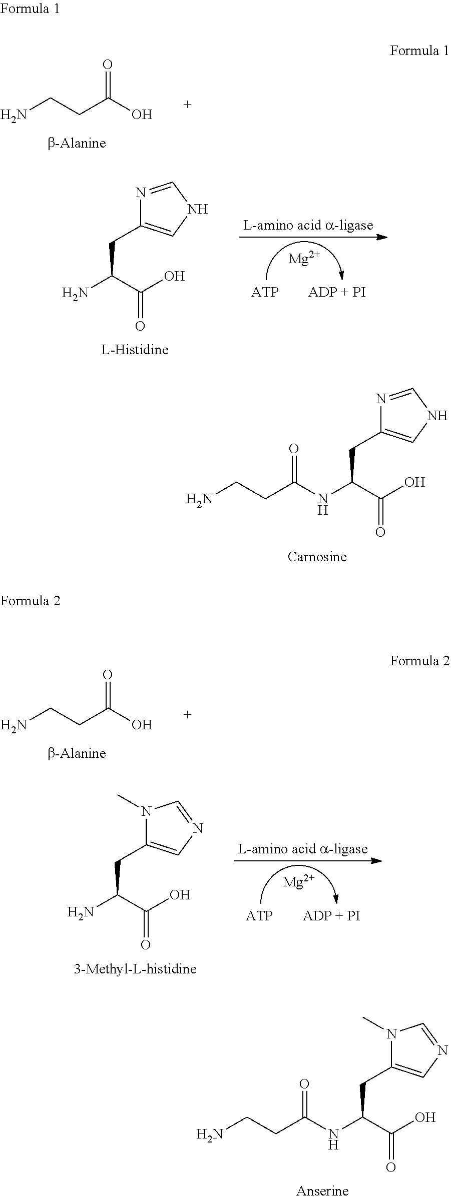

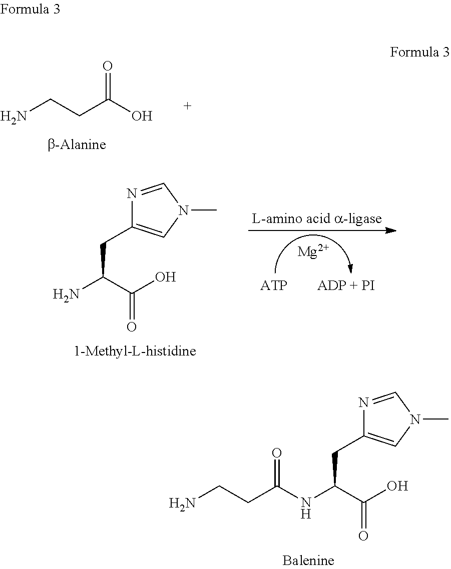 [SMILES: NCCC(=O)O ; Cn1cnc(C[C@H](N)C(=O)O)c1 ; [Mg+2] ; Cn1cnc(C[C@H](NC(=O)CCN)C(=O)O)c1]

As described herein, L-amino acid α-ligase activity refers to an activity of condensing amino acid, which is the substrate, and binding a peptide at the α-position carboxy group, of which dipeptide synthesizing activity refers to an activity for synthesizing dipeptide by condensation of two molecules of amino acids. The evaluation of dipeptide synthesizing activity can be measured using, for example, high-performance liquid chromatography by incubating the test protein in a buffer solution of, for example, pH 5 to 11, containing amino acid as the substrate and ATP at a prescribed temperature of, for example, 20-50° C., for a prescribed period of time, for example, 2-150 hours, and using at least one of the following as an index: an increase in the amount or concentration of the dipeptide that is produced by the incubation, a reduction in the amount or concentration of the amino acid as the substrate, a reduction in the amount or concentration of ATP, an increase in the amount or concentration of ADP, and an increase in the amount or concentration of inorganic phosphoric acid.

In addition, the microorganism having the imidazole dipeptide synthesizing activity can be a microorganism having the ability to produce L-histidine in addition to the imidazole dipeptide synthesizing activity. Microorganisms capable of producing L-histidine can be produced according to known methods (Japanese Laid-Open Patent Application Sho. 58 (1983)-193695, Japanese Laid-Open Patent Application Sho. 60 (1985)-024193, Japanese Laid Open Patent Application No. 2001-086998, Japanese Laid-Open Patent Application No. 2001-157596). By using a microorganism having L-histidine producing ability, carnosine can be produced and obtained without adding L-histidine as a substrate when producing carnosine.

As described herein, examples of the microorganism can be any prokaryote or eukaryote; more specific examples of the microorganism that can be suitably used include genus Escherichia bacteria, such as Escherichia coli, genus Actinomyces bacteria, genus Bacillus bacteria, genus Serratia bacteria, genus Pseudomonas bacteria, genus Corynebacterium bacteria, genus Brevibacterium bacteria, genus Rhodococcus bacteria, genus Lactobacillus bacteria, genus Streptomyces bacteria, genus Thermus bacteria, genus Streptococcus bacteria, genus Saccharomyces yeast, genus Pichia yeast, genus Kluyveromyces yeast, genus Candida yeast, genus Schizosaccharomyces yeast, genus Debaryomyces yeast, genus Yarrowia yeast, genus Cryptococcus yeast, genus Xanthophyllomyces yeast, genus Mortierella fungi, genus Fusarium fungi, and microorganisms belonging to the genus Schizochytrium and the genus Thraustochytrium.

More specifically, microorganisms that can be used in embodiments of the present invention include Escherichia coli (hereinafter referred to as “E. coli”), Bacillus subtilis, Bacillus brevis, Bacillus stearothermophilus, Serratia marcescens, Pseudomonas putida, Pseudomonas aeruginosa, Corynebacterium glutamicum, Brevibacterium flavum, Brevivacterium lactofermentum, Rhodococcus erythropolis, Thermus thermophilus, Streptococcus lactis, Lactobacillus casei, Streptomyces lividans, Saccharomyces cerevisiae, Saccharomyces bayanus, Pichia pastoris, Kluyveromyces lactis, Candida utilis, Candida glabrata, Schizosaccharomyces pombe, Debaryomyce hansenii, Yarrowia lypolitica, Cryptococcus curvatus, Xanthophyllomyces dendrorhous, Aspergillus nigar, Aspergillus oryzae, Mortierella ramanniana, Mortierella bainieri, Mortierella alpina, Cunninghamella elegans, Fusarium fujikuroi, Schizochytrium limacium, and Thraustochytrium aureum.

Preferred examples of the microorganism can include microorganisms such as Escherichia coli, actinomycetes, genus Corynebacterium bacteria, genus Bacillus bacteria, genus Pseudomonas bacteria, genus Saccharomyces yeast, of which Escherichia coli is the most preferred example of the microorganism.

As described herein, the microorganism is not necessarily limited as long as it has imidazole dipeptide synthesizing activity; for example, a transformed strain in which a mutant L-amino acid α-ligase having L-amino acid α-ligase activity derived from Bacillus subtilis is expressed in the microorganism by a known method (Japanese Laid Open Patent Application No. 2018 and Drozak J. et al., J. Biol. Chem. 2015, 290: 17190-17205) can be formed and used as a microorganism having imidazole dipeptide synthesis activity.

In brief, for example, a recombinant DNA encoding the above-described mutant L-amino acid α-ligase is prepared. For example, it can be obtained by introducing a site-specific mutation, using a primer designed from the polynucleotide sequence of SEQ ID NO: 12, and using site-directed mutagenesis in which chromosomal DNA of microorganisms encoding YwfE protein or a related protein thereof such as Bacillus subtilis 168 is used as a template.

More specifically, introduction of the desired mutation into the template gene can be carried out using various methods of site-directed mutagenesis that are well-known to a person skilled in the art, based on PCR amplification using a polynucleotide of SEQ ID NO: 12 as template DNA or a replication reaction with various DNA polymerases. The site-directed mutagenesis method can be performed, for example, by any method such as the PCR method or the annealing method (Muramatsu et al. eds., “Revised 4th Edition New Genetic Engineering Handbook”, Yodosha, p. 82-88). If necessary, various commercially available site-directed mutagenesis kits such as QuickChange II Site-Directed Mutagenesis Kit (Stratagene, USA) and QuickChange Multi Site-Directed Mutagenesis Kit (Agilent Technology, USA) can be used.

Template DNA containing the YwfE gene can be prepared from bacteria producing YwfE proteins by extracting genomic DNA or extracting RNA and synthesizing cDNA by reverse transcription by a conventional method. Bacteria producing YwfE proteins have been reported in plants and animals, in addition to bacteria including genus Bacillus bacteria such as Bacillus subtilis, genus Clostridium bacteria, and genus Acidothermus bacteria; however, a genus Bacillus bacteria such as Bacillus subtilis is preferable and can be easily obtained by a person skilled in the art.

Preparation of genomic DNA from these Bacillus bacteria can be performed, for example, by the method described in Pitchereta et al., Lett. Appl. Microbiol., 1989, 8: p. 151-156. The template DNA containing the YwfE gene can be prepared by inserting a DNA fragment containing the YwfE gene extracted from the prepared cDNA or genomic DNA into an arbitrary vector.

Introduction of a site-specific mutation into the YwfE gene can be most commonly performed using a mutation primer containing the nucleotide mutation to be introduced. Such a mutant primer can be designed to anneal to a region of the YwfE gene containing the nucleotide sequence encoding the amino acid residue to be substituted, and to include a base sequence that has a nucleotide sequence (codon) encoding the amino-acid residue after substitution in place of the nucleotide sequence (codon) encoding the amino acid residue to be substituted.

Using recombinant DNA obtained by these methods, a vector for causing a host strain to express the following protein of embodiments of the present invention is prepared. Expression vectors for producing recombinant DNA are commercially available. By obtaining and utilizing these vectors, the nucleic acid insertion vector as described herein can be prepared and used for the preparation of the following host strains containing the present nucleic acid insertion vector, i.e., transformants.

When E. coli is used as the host strain, examples of vectors include pColdI (manufactured by Takara Bio), pCDF-1b, pRSF-1b (both manufactured by Novagen), pMAL-c2x (manufactured by New England Biolabs), pGEX-4T-1 (manufactured by GE Healthcare Bioscience), pTrcHis (manufactured by Invitrogen), pSE280 (manufactured by Invitrogen), pGEMEX-1 (manufactured by Promega), pQE-30 (manufactured by Qiagen), pET-3 (manufactured by Novagen), pBluescriptll SK (+), pBluescript II KS (−) (manufactured by Stratagene), pTrS30 [prepared from Escherichia coli JM109/pTrS30 (FERM BP-5407)], and the like.

When the vector is used, any promoter can be used as long as it functions in a host strain such as E. coli, examples of which include promoters derived from E. coli, phage, etc., such as trp promoter (Ptrp), lac promoter (Plac), PL promoter, PR promoter, and PSE promoter. When a microorganism belonging to the genus Bacillus is used as the host strain, an SPO1 promoter, an SPO2 promoter, a penP promoter, or the like, which function in Bacillus subtilis can also be used. In addition, artificially designed and modified promoters such as a promoter in which two Ptrps are connected in series, a tac promoter, a lacT7 promoter, or a let I promoter can also be used.

When a vector is used for producing the protein described herein, an expression vector can be particularly useful. The expression vector is not necessarily limited as long as it is a vector that expresses a protein in a test tube, in Escherichia coli, in cultured cells, or in the individual organism, but preferably is a pBEST vector (manufactured by Promega) if it is expressed in a test tube, a pET vector (manufactured by Invitrogen) in the case of E. coli, a pME18S-FL3 vector (GenBank Accession No. AB009864) in the case of cultured cells, and a pME18S vector (Mol. Cell Biol. 8: 466-472 (1988)) in the case of an individual organism. Insertion of the DNA of embodiments of the present invention into a vector can be carried out by a conventional method, for example, by a ligase reaction using a restriction enzyme site (Current Protocols in Molecular Biology edited by Ausubel et al. (1987) Publish. John Wiley & Sons. Section11.4-11.11).

Introduction of the nucleic acid-inserted vector into the host strain can be carried out by calcium phosphate transfection, DEAE-dextran mediated transfection, polypropylene mediated transfection, protoplast fusion, liposome mediated transfection (lipofection), conjugation, natural transformation, electroporation, and other methods known to those skilled in the art. In addition, by obtaining a commercially available transfection reagent and using these, the expression vector can be introduced into a host strain. [Reference: Current Protocols in Molecular Biology. 3 Vols. Edited by Ausubel F M et al., John Wiley & Son, Inc., Current Protocols.]

The production of imidazole dipeptide is carried out, for example, using a transformant expressing a mutant L-amino acid α-ligase as a microorganism having L-amino acid α-ligase activity, mixing the transformant with substrates of the reaction formula represented by formulas 1 to 3, i.e., β-alanine and L-histidine when producing carnosine, β-alanine and 3-methyl-L-histidine when producing anserine, and β-alanine and 1-methyl-L-histidine when producing balenine, and further mixing, for example, a glucide, using the ATP produced by the microorganisms metabolizing the glucide as an ATP supply system, and using a bacterial reaction method or a fermentation method to produce the desired imidazole dipeptide.

That is, with the production method described herein, it is possible to produce the desired imidazole dipeptide, such as carnosine, anserine, and balenine, without including the addition of ATP, or by adding an amount of ATP smaller than the amount of imidazole dipeptide produced in terms of number of moles.

The term “bacterial reaction method” refers to a method for producing a target product using a microorganism, and the enzyme activity inside the microorganism in a state where growth is stopped or stopped is measured, and does not require a raw material of a culture medium for growth or propagation. In the present specification, the term “fermentation method” refers to a method for producing a target product using microorganisms that are in a growing or propagating state.

Examples of the substrate in the ATP supply system are not limited to the glucide, and are not particularly limited as long as the microorganism can utilize the substrate to produce ATP. Specific examples include carbohydrates and lipids. Examples of carbohydrates include glucides (sugars/monosaccharides/disaccharides), trisaccharides (oligosaccharides), and sugar alcohols (glycerol and the like). Preferable examples are glucose and glycerol. Examples of lipids include fatty acids such as palmitic acid and stearic acid.

Examples of embodiments in which an imidazole dipeptide is produced by a bacterial reaction method or a fermentation method in the production method of the present invention aew described in more detail below.

To produce the desired imidazole dipeptide according to the production method of embodiments of the present invention, for example, a microorganism having imidazole dipeptide synthesizing activity is used, an amino acid which is a raw material serving as a substrate of the protein described herein is used as a substrate in an ATP supply system, and bacterial cell reaction is carried out in the presence of a glucide in a buffer solution, by adjusting the pH appropriately without using a buffer, or by culturing cells in a cell culture medium. These dipeptides can be produced by isolating the desired dipeptide from a buffer solution, a reaction solution containing no buffer, or a culture solution after this reaction. Examples of the amino acid as a raw material include a combination of β-alanine (β-Ala) and one amino acid selected from L-histidine or L-histidine derivative, preferably, β-Ala and L-His, β-Ala and 3-methyl-L-histidine, or B-Ala and 1-methyl-L-histidine.

Examples of the buffer solution include phosphate buffer, borate buffer, citrate buffer, acetate buffer, and tris-HCl buffer, commonly used by those skilled in the art. On the other hand, in the production method of embodiments of the present invention, the imidazole dipeptide can be produced without using a buffer by appropriately adjusting the pH of the reaction solution or the culture solution.

For the carbon source components contained in the cell culture medium, any carbon source can be used as long as the carbon source can be assimilated by the microorganism having imidazole dipeptide synthesizing activity of embodiments of the present invention; such carbon sources include glucose, fructose, sucrose, carbohydrates containing these sugars such as molasses, starch, and starch hydrolyzates, organic acids such as acetic acid and propionic acid, and alcohols such as ethanol and propanol.

Examples of the nitrogen source that can be used include ammonium salt of inorganic or organic acids such as ammonia, ammonium chloride, ammonium sulfate, ammonium acetate, and ammonium phosphate, other nitrogen-containing compounds, as well as peptone, meat extract, yeast extract, corn steep liquor, and casein hydrolyzate, soybean meal, soybean meal hydrolyzate, various fermentation cells, and digests thereof.

Examples of the inorganic salt that can be used include potassium phosphate, dipotassium phosphate, magnesium phosphate, magnesium sulfate, sodium chloride, ferrous sulfate, manganese sulfate, copper sulfate, and calcium carbonate. In addition, nutrients such as peptone, meat extract, yeast extract, corn steep liquor, casamino acids, and various vitamins such as biotin can be added to the medium.

Cultivation is usually carried out under aerobic conditions such as aeration stirring and shaking. The culture temperature is not particularly limited as long as the microorganism having imidazole dipeptide synthesizing activity of the present invention can grow, and the pH during the culturing is also not particularly limited as long as the pH is that in which the microorganism having imidazole dipeptide synthesizing activity of the present invention can grow. The pH adjustment during the culture can be carried out by adding an acid or an alkali.

An example of the production method when the imidazole dipeptide is carcinone, anserine, and balenine is described more specifically below.

Method for Producing Carnosine

Carnosine is produced by a condensation reaction using β-alanine and L-histidine as the substrate, and a microorganism having carnosine synthesizing activity, typically a microorganism expressing a mutant YwfE having an amino-acid sequence represented by SEQ ID NO: 13 to 32 as a biocatalyst. Therefore, microorganisms having carnosine synthesizing activity, typically a mutant YwfE having an amino acid sequence represented by SEQ ID NOs: 13 to 32, are made to coexist in a buffer solution containing β-alanine and L-histidine, a glucide such as glucose, and magnesium sulfate, and are reacted for a prescribed period of time, after which the produced carnosine is isolated and purified to thereby produce the desired carnosine. Alternatively, carnosine can be produced by carrying out the reaction while appropriately adjusting the pH of the reaction solution or culture solution without using a buffer solution. The glucide to be added to the reaction solution or the culture solution is typically glucose, and the amount of glucose to be added is a concentration of 0.001 to 3 mol/L, preferably 0.005 to 2 mol/L, and more preferably 0.01 to 1 mol/L.

The carnosine formation reaction is carried out in an aqueous medium under conditions of pH 5 to 11, preferably pH 6 to 10, at a temperature of 20 to 45° C., preferably 25 to 40° C., for 2 to 72 hours, preferably 6 to 36 hours.

Isolation and purification of carnosine generated and accumulated in a buffer solution, or a culture solution or a reaction solution containing no buffer, can be carried out using a method commonly used by those skilled in the art using activated carbon or ion exchange resin, or through extraction with an organic solvent, crystallization, thin layer chromatography, high-performance liquid chromatography, or the like.

Method for Producing Anserine

Anserine is a peptide in which β-alanine and 3-methyl-L-histidine are peptide-bonded. Therefore, when anserine is produced using a microorganism having anserine synthesizing activity, typically a microorganism expressing mutant YwfE having an amino acid sequence represented by SEQ ID NOs: 13 to 32, besides using 3-methyl-L-histidine instead of L-histidine, which is one of the substrates described above in “Method for producing carnosine,” incubation can be carried out using the same conditions as those described in “Method for producing carnosine” to isolate and purify the synthesized anserine, to thereby produce and obtain anserine.

Method for Producing Balenine

Balenine is a peptide in which β-alanine and 1-methyl-L-histidine are peptide-bonded. Therefore, when balenine is produced using a microorganism having balenine synthesizing activity, typically a microorganism expressing mutant YwfE having an amino acid sequence represented by SEQ ID NOs: 13 to 32, besides using 1-methyl-L-histidine instead of L-histidine which is one of the substrates as described above in “Method for producing carnosine,” production can be carried out using the same conditions as those described in “Method for producing carnosine” to produce and obtain balenine.

Production Method Using a Peptidase-Deficient Strain of an Imidazole Dipeptide-Degrading Enzyme

Imidazole dipeptide is degraded by peptidase contained in microorganisms such as E. coli. Therefore, in order to produce imidazole dipeptide in high yields using a microorganism, production can be carried out by preparing and using a peptidase-deficient strain. For the preparation of the above-described peptidase-deficient strain, a commercially available chromosome modification system can be used. For example, when a peptidase-deficient strain of E. coli is prepared, a commercially available kit for modifying the E. coli chromosome, such as the Quick and Easy E. coli Gene Deletion Kit, can be obtained and used. For example, a strain in which the host is provided with a peptidase deficiency, such as a strain in which an Escherichia coli strain is given a peptidase PepD deficiency, can be used.

The imidazole dipeptides such as carnosine, anserine and balenine produced by the above-described production method can be mixed, for example, with pharmaceutical additives and/or food additives to produce tablets, capsules, liquids, and the like by means of well-known and conventional methods, and used as a pharmaceutical drug or functional food/food supplement for preventing or treating disease based on their anti-fatigue effects, ability to scavenge active oxygen and lower blood pressure, anti-inflammatory effects, or uric acid level lowering effects.

Method for Producing Anserine from Carnosine

Another embodiment of the present invention is a method for producing anserine from carnosine.

That is, it is a method for producing anserine, which is one type of imidazole dipeptide, in which the imidazole dipeptide is carnosine, comprising further reacting carnosine produced by the microorganism, or carnosine obtained by extracting from the muscle of chicken, etc., with an enzyme having carnosine N-methyltransferase activity.

More specifically, anserine can be produced from carnosine by using a microorganism expressing an enzyme having carnosine N-methyltransferase activity.

In addition, by an embodiment of the present invention, a microorganism coexpressing an enzyme having carnosine synthesizing activity and an enzyme having carnosine N-methyltransferase activity can be used as the L-amino acid α-ligase to thereby produce anserine.

In the present Specification, carnosine N-methyltransferase activity refers to enzyme activity for producing anserine (anserine: β-alanyl-3-methyl-L-histidine) by transferring the histidine residue of carnosine to the 3-methylhistidine residue, using S-adenosylmethionine (SAM) as the methyl group donor.

The enzyme having carnosine N-methyltransferase activity used in this embodiment can be obtained by cloning using a PCR method or the like, well-known to those skilled in the art.

A specific example of the enzyme having the carnosine N-methyltransferase activity used in the present embodiment is a peptide having carnosine N-methyltransferase activity derived from Saccharomyces cerevisiae (YNL092W: SEQ ID NO: 36).

More preferable examples of the peptide include:

(i) a peptide having an amino-acid sequence of SEQ ID NO: 36,
(ii) a peptide having a homology of 80% or more, preferably 85% or more, more preferably 90% or more, and most preferably 95% or more with an amino-acid sequence of SEQ ID NO: 36, and that has carnosine N-methyltransferase activity, and
(iii) a peptide comprising an amino-acid sequence in which one or a plurality of amino-acid residues of an amino-acid sequence of SEQ ID NO: 36 are deleted, substituted, inserted, and/or added, and that has carnosine N-methyltransferase activity.

In this embodiment, the production method can be selected from a bacterial reaction method or a fermentation method; however, although not required, a fermentation method is preferred.

The bacterial reaction method and the fermentation method can be carried out in accordance with the same method as described in the above-mentioned “Method for producing imidazole peptide.”

In the bacterial reaction method and the fermentation method, the SAM as a methyl group donor can be synthesized from ATP and L-Met by methionine adenosyltransferase (AMT) in vivo. SAM can be produced from D-glucose-derived ATP and L-Met generated by metabolism in the bacteria cells or microorganisms. Therefore, anserine can be produced from carnosine without adding SAM by using a bacterial reaction method or a fermentation method. That is, in the bacterial reaction method or fermentation method, by adding carnosine, D-glucose, and L-methionine to a microorganism having carnosine N-methyltransferase activity, anserine can be produced without adding SAM or ATP.

A microorganism described in the method for producing imidazole dipeptide can be used as the microorganism used in the present embodiment; however, Escherichia coli, although not required, is preferable. A method using Escherichia coli as a host is common as a substance production system, and the expression of a eukaryote-derived enzyme gene in Escherichia coli enables higher expression, and can provide an efficient method of producing the target useful substance by cooperation with other related enzymes from E. coli.

In addition, high expression of a variant of the amino acid ligase YwfE derived from a prokaryotic microorganism found by the present inventors can be achieved by using Escherichia coli instead of yeast as the host (Japanese Laid Open Patent Application No. 2018-102287). Therefore, when the above-described co-expression system of carnosine synthesizing enzyme and carnosine N-methyltransferase is used, Escherichia coli can be used as the host.

The anserine produced in the present embodiment can be used as a food or medicine that promote health, since anserine has an anti-fatigue effect and a uric acid level lowering effect.

All documents mentioned in the present Specification are incorporated by reference in their entirety. The examples described herein are illustrative of embodiments of the present invention and should not be construed as limiting the scope of the invention.

### Example 1

In the following examples, preparation of polynucleotides (DNA, mRNA), PCR, base sequence determination, transformation, HPLC analysis, and the like can be carried out using conventional methods well known to those skilled in the art. Refer to, for example, Sambrook, J. and Russell, D W, Molecular Cloning, A Laboratory Manual 3rd Edition, Cold Spring Harbor Laboratory Press (2012), and the like.

Introduction of Site-Specific Mutation into YwfE

Using a pET vector into which YwfE gene (SEQ ID NO: 1) was incorporated as a template, the desired mutation was introduced using Quick Change Site-Directed Mutagenesis (Strategene, USA) following the manufacturer's instructions. A PCR reaction was carried out under the reaction conditions shown in Table 1 (composition) and Table 2 (PCR cycle). In the PCR reaction, KOD-Plus-Neo-DNA polymerase (Toyobo Co., Ltd., Osaka) was used. Primers (SEQ ID NOs: 2 to 5) were used to obtain a vector into which a site-specific mutation of N108E or I112V was introduced. The vector for N108E represents a polynucleotide (SEQ ID NO: 6) encoding a YwfE protein in which the 108th asparagine (N) residue of the YwfE protein has been substituted with a glutamic acid (E) residue, and the vector for I112V represents a polynucleotide (SEQ ID NO: 7) encoding a YwfE protein in which the 112th isoleucine (I) residue of the YwfE protein has been substituted with a valine (V) residue.

TABLE 1

| | | |
|---|---|---|
| KOD-Plus-Neo buffer | 5 | μL |
| 2 mM dNTP | 5 | μL |
| 25 mM MgS04 | 3 | μL |
| 10 mM forward primer | 1 | μL |
| 10 mM reverse primer | 1 | μL |
| Template plasmid (50 μg/mL) | 1 | μL |
| KOD-Plus-Neo-DNA polymerase | 1 | μL |
| Sterile MilliQ | 33 | μL |
| Total | 50 | μL |

TABLE 2

| Step | Temperature/° C. | Time/sec |
|---|---|---|
| Thermal denaturation before cycle | 94 | 120 |
| Thermal denaturation | 98 | 10 |
| Annealing, extension | 68 | 210 |
| Last extension reaction | 68 | 60 |
| Storage | 4 | ∞ |

Number of PCR reaction (thermal denaturation/annealing, extension) cycles: 40

After the PCR reaction, the obtained PCR product was analyzed with a DNA sequencer (Applied Biosystems, Life Technologies Japan Co., Ltd., Tokyo) to confirm whether a site-specific mutation was introduced.

Although the vector extracted from Escherichia coli, the Dpn I site is methylated by Dam methylase, the Dpn I site is not methylated in the PCR product; therefore, the use thereof makes it is possible to distinguish between the template vector and the PCR product. In short, purified PCR product was treated with DpnI at 37° C. for 2 hours, in order to remove the template vector contained in the reaction solution after PCR. The restriction enzyme reaction was carried out under the reaction conditions shown in Table 3.

TABLE 3

| | | |
|---|---|---|
| DNA (PCR product) | 50 | μg |
| 10 × TA buffer | 5 | μL |
| Dpn I (1000 U) | 1 | μL |
| MilliQ | 44 | μL |

After digestion with Dpn I, the site-directed mutagenized vector purified by phenol-chloroform treatment and ethanol precipitation was dissolved in TE buffer (Tris-EDTA Buffer) pH 8.0.

Transformation of Escherichia coli Host with Site-Directed Mutagenesis Vector

Both competent cells of Escherichia coli BL21 (DE3) and the site-directed mutagenesis vector were heat-treated at 42° C. Thereafter, SOC (Super Optimal broth with Catabolite repression) medium was added and cultured, the cells were inoculated on LB agar medium containing 50 μg/mL kanamycin, and cultured at 37° C. overnight. The recombinant Escherichia coli which formed the colonies was used as a site-directed mutagenized YwfE-expressing transformant.

One colony was selected from the grown colonies, which was suspended in 3 mL of LB medium containing kanamycin, and cultured at 37° C. for 5 hours. After the culture, a site-directed mutagenesis vector was extracted from the transformed strain by means of the alkaline SDS method. The extracted site-directed mutagenesis vector was purified by phenol/chloroform treatment and ethanol precipitation, and dissolved in TE buffer (pH 8.0).

Transformation of Escherichia coli PepD-Deficient Strain with Site-Directed Mutagenesis Vector

Using the purified site-directed mutagenesis vector, the Escherichia coli PepD-deficient strain was transformed by the heat shock method. Both competent cells of E. coli PepD-deficient strain and the site-directed mutagenesis vector were heat-treated at 42° C. Thereafter, SOC medium was added and cultured, the cells were inoculated on LB agar medium containing 50 μg/mL kanamycin, and cultured at 37° C. overnight. The recombinant Escherichia coli PepD-deficient strain which formed the colonies was used as a site-directed mutagenized YwfE-expressing PepD-deficient transformant.

Double Mutant Enzyme-Expressing Transformant

In Escherichia coli BL21 (DE3) and PepD-deficient strains, a double mutant enzyme N108E/H378K (SEQ ID NO: 8) and I112V/H378K (SEQ ID NO: 9)-expressing E. coli, which combines the mutation of N108E or I112V and the mutation of H378K, was prepared and the carnosine synthesizing activity was evaluated.

In brief, as in the case of the introduction of the site-specific mutation described above, the vector having the site-specific mutation of N108E or I112V was turned into a template, and the site-specific mutation (H378K) was further introduced using primers of H378K (SEQ ID NOs: 10 and 11) to obtain a vector having a double site-specific mutation. Thereafter, in the same manner as described above, the expressed transformant into which the purified double mutant YwfE (SEQ ID NOs: 23 and 28) was introduced was obtained.

Evaluation of Carnosine Synthesizing Activity

HPLC Analysis

Commercial carnosine (Sigma-Aldrich, USA) was used as the carnosine sample. The synthesized amount of the peptide was analyzed by HPLC using Nα-(5-fluoro-2,4-dinitrophenyl)-L-alanine amide (FDAA) derivatization method and quantified by means of the calibration curve method. The HPLC analysis was carried out in accordance with a standard method under the conditions shown in Table 4 (eluent composition) and Table 5 (gradient program).

Analysis conditions

Equipment used: HITACHI L-7000 series (Hitachi, Ltd., Tokyo)

Column used: WH-C18A (4×150 mm) (Hitachi High-Technologies Corporation, Tokyo)

Sample injection volume: 10 μL

Flow rate: 0.5 mL/min

Column temperature: 40° C.

UV detection wavelength: 340 nm

TABLE 4
Eluent composition in HPLC analysis

| | Eluent | Eluent | Eluent |
|---|---|---|---|
| Component | A (mL) | B (mL) | C (mL) |
| Acetonitrile | 50 | 350 | 600 |
| Methanol | 50 | 50 | — |
| Tetrahydrofuran | — | — | 200 |
| 50 mM KH₂P0₄ (pH 2.7) | 900 | 600 | — |
| MilliQ | — | — | 200 |
| Total volume | 1000 | 1000 | 1000 |

TABLE 5
Eluent gradient conditions in HPLC

| Time (minutes) | Eluent A (%) | Eluent B (%) | Eluent C (%) |
|---|---|---|---|
| 0 | 80 | 20 | 0 |
| 10 | 80 | 20 | 0 |
| 35 | 0 | 100 | 0 |
| 35.1 | 0 | 0 | 100 |
| 37.1 | 0 | 0 | 100 |
| 37.2 | 80 | 20 | 0 |
| 50 | 80 | 20 | 0 |

Evaluation of Effect of Peptidase on Carnosine Degradation

Using Escherichia coli BL21 (DE3) (Wild type) and a PepD-deficient strain (ΔPepD), the effect of peptidase PepD on carnosine degradation was evaluated. Carnosine was prepared to have an initial concentration of 5.0 mM and a wet bacterial mass of 10 mg/ml, and reacted at 30° C. for 20 hours.

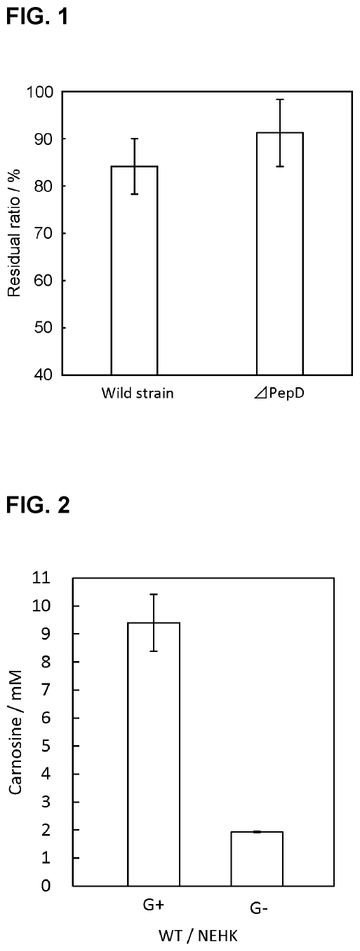

As a result, the residual rate of carnosine was 84.1% when Escherichia coli BL21 (DE3) was used and 91.3% when the defective strain was used (FIG. 1).

Therefore, it was revealed that in the PepD-deficient strain, the degradation of carnosine by PepD was suppressed.

Carnosine Synthesis by Means of Bacterial Reaction Method

Carnosine synthesis reaction in a double mutant YwfE (N108E/H378K)-expressing transformant strain using Escherichia coli BL21 (DE3) (wild type) as a host was carried out in a reaction solution having the composition shown in Table 6, at a temperature of 30° C. and pH of 7.0 to 8.0 for 20 hours. After completion of the reaction, the double mutant YwfE (N108E/H378K)-expressing transformant was analyzed by HPLC in the same manner as described above to evaluate the carnosine synthesizing activity thereof.

TABLE 6

| Components | Composition | |
|---|---|---|
| β-Ala | 12.5 | mM |
| L-His | 12.5 | mM |
| D-Glucose | 50.0 | mM |
| MgSO₄ | 12.5 | mM |
| Whole cell | 10 | mg/ml |
| HEPES Buffer (pH 8.0) | 100 | mM |
| Total | 300 | μL |

Results

As a result, the carnosine synthesizing activity was 1.9 mM in the glucose-free group (G−), whereas the synthesized carnosine concentration increased to 9.4 mM in the glucose-added group (G+) (FIG. 2).

Therefore, it is that carnosine can be efficiently synthesized by adding glucose without adding the substrate ATP.

Evaluation of Effect of pH Control in Bacterial Reaction Method

In the bacterial reaction method described above, as shown in Table 7, the HEPES buffer was changed to an aqueous solution, glucose was added, the reaction solution was adjusted to pH 8.0 and the volume thereof to 100 mL at the start of the reaction, and reaction was carried out in the same manner as the above-described bacterial reaction method, while comparing a case in which the reaction pH was not controlled and a case in which the lower limit value of the pH was controlled to pH 7.7 with NaOH.

TABLE 7

| Components | Composition | |
|---|---|---|
| β-Ala | 12.5 | mM |
| L-His | 12.5 | mM |
| D-Glucose | 50.0 | mM |
| MgSO₄ | 12.5 | mM |
| Whole cell | 100 | mg/ml |
| HEPES Buffer (pH 8.0) | 100 | mM |
| Total | 100 | mL |

Results

In the case of carnosine synthesis when the pH was not controlled, the reaction solution dropped to pH 6.6 after 20 hours of reaction time, and the synthesized carnosine concentration was 8.8 mM, whereas when the lower limit of pH was controlled to pH 7.7, the concentration of synthesized carnosine was 12.5 mM.

Therefore, when glucose was added without adding the substrate ATP, it was clear that carnosine can be efficiently synthesized by controlling the pH even if a buffer solution is not used and changed to an aqueous solution.

### Example 2

Anserine Synthesis by Means of Bacterial Reaction Method

Anserine synthesis in a double mutant YwfE (I112V/H378K)-expressing transformant strain using Escherichia coli BL21 (DE3) (wild type) and a PepD-deficient strain (ΔPepD) of said E. coli as a host was carried out in a reaction solution having the composition shown in Table 7, at a temperature of 30° C. and pH of 7.0 to 8.0 for 20 hours. Following reaction, anserine synthesizing activity was evaluated by quantifying the anserine concentration by HPLC in the same manner as in Example 1. In addition, the anserine sample was prepared using commercially available anserine (Wako Pure Chemical Industries, Ltd., Osaka), so that the wet bacterial mass was 10 mg/ml.

TABLE 8

| Components | Composition | |
|---|---|---|
| β-Ala | 12.5 | mM |
| 3-Methyl-L-His | 12.5 | mM |
| D-Glucose | 50.0 | mM |
| MgSO₄ | 12.5 | mM |
| Whole cell | 10 | mg/mL |
| HEPES Buffer (pH 8.0) | 100 | mM |
| Total | 300 | μL |

Results

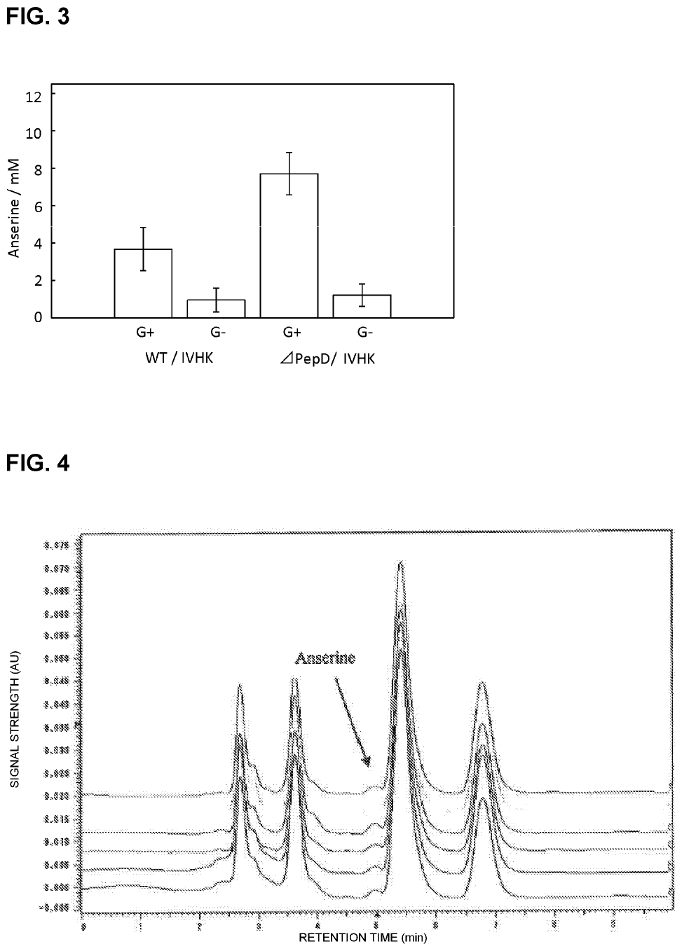

When comparing between the glucose-free group (G−) and the glucose-added group (G+), in the transformant in which double mutant YwfE (I112V/H378K) was expressed using Escherichia coli BL21 (DE3) and the PepD-deficient strain thereof as a host, the anserine synthesizing activity respectively increased from an anserine concentration of 1.0 mM to 3.7 mM, and from 1.2 mM to 7.7 mM (FIG. 3.

Therefore, it is clear that anserine can be efficiently synthesized by adding glucose without adding substrate ATP. It also is clear that the anserine synthesizing activity of the transformant expressing the double mutant YwfE (I112V/H378K) using the PepD-deficient strain as a host is increased by the addition of glucose while the degradation of anserine is suppressed.

From these results, even with regard to balenine, it was determined that the production reaction in the bacterial reaction method or the fermentation method using a microorganism having balenine synthesizing activity enables efficient synthesis by coupling ATP production from glucose, without adding substrate ATP.

### Example 3

Cloning of Carnosine N-Methyltransferase

Carnosine N-methyltransferase (YNL092W) (SEQ ID NO: 35) was cloned by PCR using genomic DNA extracted from Saccharomyces cerevisiae (X33) as a template. PCR reaction was carried out under the reaction conditions shown in Table 9 (composition) and Table 10 (PCR cycle). In the PCR reaction, KOD One™ PCR Master Mix Toyobo Co., Ltd., Osaka) and primers (SEQ ID NOs: 33 to 34) (Table 11) were used.

TABLE 9

| | | |
|---|---|---|
| KOD One ™ PCR Master Mix | 25 | μL |
| 10 μM forward primer | 1.5 | μL |
| 10 μM reverse primer | 1.5 | μL |
| Template genomic DNA (50 μg/mL) | 1 | μL |
| Sterile MilliQ | 21 | μL |
| Total | 50 | μL |

TABLE 10

| | |
|---|---|
| Denaturation | 98° C., 10 sec |
| Annealing | 50° C., 5 sec |
| Extension | 68° C., 6 sec |

Number of cycles: 40 cycles

TABLE 11

| | | Restriction |
|---|---|---|
| Primer | Sequence | enzyme |
| Forward | CCCGGGCATATGGACGAGAAT | NdeI |
| (SEQ ID NO: 33) | GAATTTGAT | |
| Reverse | ATAGCGGCCGCTGATTCATTG | NotI |
| (SEQ ID NO: 34) | GTGGGGT | |

Agarose electrophoresis was carried out on the PCR amplification product, and a band having the same size as the target product was extracted and purified. The obtained PCR amplification product was inserted into a pET21a (+) vector. At this time, it was designed such that a His tag is added to the C-terminal side of the expressed carnosine N-methyltransferase (YNL092W) (SEQ ID NO: 36).

After the PCR reaction, the obtained PCR product was analyzed with a DNA sequencer (Applied Biosystems, Life Technologies Japan Co., Ltd., Tokyo) to confirm whether the target carnosine N-methyltransferase (YNL092W)-expressing vector was obtained.

Transformation of Escherichia coli Host with Expression Vector

Both the competent cells of Escherichia coli BL21 (DE3) and the carnosine N-methyltransferase (YNL092W)-expressing vector were heat-treated at 42° C. Thereafter, 200 μL of the culture solution was applied to an LB agar medium containing 100 μg/mL of ampicillin, and cultured overnight. Thereafter, colonies (transformants) that grew on the agar medium were inoculated into 3 mL of an LB medium containing 100 μg/mL of ampicillin, and cultured at 37° C. for 5 hours.

Induction of carnosine N-methyltransferase (YNL092W) expression in transformants 2 mL of the culture solution obtained from the above culture was inoculated into 200 mL of an LB medium containing 100 μg/mL of ampicillin, and when the OD₆₀₀ value reached 0.5, IPTG (isopropyl-β-thiogalactopyranoside: final concentration 100 μM) was added, to induce expression of carnosine N-methyltransferase (YNL092W).

Bacterial Collection/Washing

After culturing, the culture was centrifuged (5,000×g, 10 minutes, 4° C.) to collect the cells, and the precipitate was washed with physiological saline.

Bacterial Disruption

After washing, the bacteria were disrupted using BugBuster HT Protein Extarction Reagent (Novagen) to obtain a bacterial cell lysate. Thereafter, the bacterial cell lysate was centrifuged (7,000×g, 30 minutes, 4° C.) to obtain a cell-free extract.

Enzyme Purification

The obtained cell-free extract was purified with a Ni affinity column HisGraviTrap and PD-10 column manufactured by GE Healthcare to obtain a purified enzyme solution. At the time of purification, the Binging buffer (Tris 50 mM, NaCl 500 mM, Imidazole 10 mM in MilliQ at pH 8.0) and an Elution buffer (Tris 50 mM, NaCl 500 mM, Imidazole 500 mM in MilliQ at pH 8.0) were used.

Enzymatic Reaction (Anserine Synthesis)

Using the obtained purified enzyme solution, the reaction solutions shown in Table 12 (composition) and Table 13 (composition) were prepared, and the enzymatic reaction was carried out at a temperature of 37° C. and a pH of 7.0 to 8.0 for 4 hours or 20 hours.

TABLE 12

| Component | Final concn. | |
|---|---|---|
| Carnosine | 2.0 | mM |
| S - adenosylmethionine | 2.0 | mM |
| MgSO₄ | 2.0 | mM |
| YNL092W | 0.50 | mg/mL |
| HEPES (pH 7.5) | 100 | mM |
| Total | 300 | μL |

TABLE 13

| Component | Final concn. | |
|---|---|---|
| Carnosine | 2.0 | mM |
| S - adenosylmethionine | 2.0 | mM |
| MgSO₄ | 0 | mM |
| YNL092W | 0.50 | mg/mL |
| HEPES (pH 7.5) | 100 | mM |
| Total | 300 | μL |

Evaluation of Anserine Synthesis by Enzyme Reaction Method

After the reaction was completed, the anserine synthesizing activity was evaluated by quantifying the anserine concentration by means of HPLC in the same manner as in Examples 1 and 2. The anserine sample was prepared using commercially available anserine (Wako Pure Chemical Industries, Ltd., Osaka) so that the wet bacterial mass was 10 mg/ml.

Anserine Synthesis by Bacterial Reaction Method

By the same operation as the above-described transformation, a transformant expressing carnosine N-methyltransferase (YNL092W) using Escherichia coli BL21 (DE3) (wild type) as a host was obtained. Anserine synthesis in the expressed transformant was carried out in reaction solutions having the compositions shown in Tables 14 and 15 at a temperature of 30° C. and pH of 7.0 to 8.0 for 20 hours. After completion of the reaction, the expressed transformant was analyzed by means of HPLC in the same manner as described above to evaluate the anserine synthesizing activity.

TABLE 14

| Component | Final concn. | |
|---|---|---|
| Carnosine | 5.0 | mM |
| d-Glucose | 20.0 | mM |
| l-Methionine | 20.0 | mM |
| MgSO₄ | 5.0 | mM |
| Whole cell | 10.0 | mg/mL |
| HEPES (pH 7.5) | 100 | mM |

TABLE 15

| Component | Final concn. | |
|---|---|---|
| Carnosine | 5.0 | mM |
| d-Glucose | 20.0 | mM |
| 1-Methionine | 20.0 | mM |
| MgSO₄ | 0 | mM |
| Whole cell | 10.0 | mg/mL |
| HEPES (pH 7.5) | 100 | mM |
| Total | 300 | μL |

Results

The amount of anserine synthesized in the enzymatic reaction method was 0.085 mM at 4 hours of reaction and 0.14 mM at 20 hours of reaction. Therefore, it was confirmed that carnosine N-methyltransferase (YNL092W) has anserine synthesizing activity.

In addition, FIG. 4 is an HPLC chromatogram showing an evaluation of anserine synthesis in a bacterial reaction method using a transformant expressing carnosine N-methyltransferase (YNL092W). Of the six chromatograms in FIG. 4, the lower three are chromatograms of reaction solutions to which MgSO₄ was added, and the upper three are chromatograms of reaction solutions to which MgSO₄ was not added. In each of the reaction solutions, a (slight) peak was confirmed at the same retention time as that of anserine (4.97 minutes), the synthesized amount of anserine was 0.042 mM, and the yield was 0.85%. Therefore, it was revealed that anserine can be synthesized from carnosine in the bacterial cell reaction method using a transformant expressing carnosine N-methyltransferase (YNL092W).

By the production method of the present invention, it is possible to produce imidazole dipeptide, particularly carnosine, anserine, and/or balenine, efficiently and at low cost, without the need for the addition of substrate ATP, which would be expensive in actual production, by coupling with ATP supply from glucose. The aforementioned imidazole dipeptides can be used as a pharmaceutical drug and/or functional food/food supplement for preventing or treating disease based on their anti-fatigue effects, ability to scavenge active oxygen and reduce blood pressure, anti-inflammatory effects, or uric acid level lowering effects.

What is claimed:

1. A method for producing imidazole dipeptide using a microorganism having imidazole dipeptide synthesis activity,
the production method not comprising adding ATP, or comprising adding an amount of ATP that is less than the amount of imidazole dipeptide that is produced in terms of the number of moles, by utilizing an ATP supply system of the microorganism.

2. The method for producing imidazole dipeptide according to claim 1, wherein the imidazole dipeptide is selected from carnosine, anserine, or balenine.

3. The method for producing imidazole dipeptide according to claim 1, wherein the microorganism is a microorganism expressing L-amino acid α-ligase.

4. The method for producing imidazole dipeptide according to claim 3, wherein the L-amino acid α-ligase is a protein having imidazole dipeptide synthesizing activity.

5. The method for producing imidazole dipeptide according to claim 3, wherein
the L-amino acid α-ligase,
is a mutant L-amino acid α-ligase in which are substituted 1 to 3 amino-acid residues of a wild-type YwfE amino-acid sequence that has an amino-acid sequence represented by SEQ ID NO: 12, and includes
a substitution of an amino-acid residue corresponding to the 108th asparagine (N) residue from the N-terminus of SEQ ID NO: 12 with an amino acid selected from alanine (A), glutamic acid (E), and glutamine (Q),
a substitution of an amino-acid residue corresponding to the 112th isoleucine (I) residue from the N-terminus of SEQ ID NO: 12 with valine (V),
or
a substitution of an amino-acid residue corresponding to the 378th histidine (H) residue from the N-terminus of SEQ ID NO: 12 with an amino acid selected from lysine (K) or arginine (R).

6. The method for producing imidazole dipeptide according to claim 1, wherein the production method includes a bacterial reaction method or a fermentation method.

7. The method for producing imidazole dipeptide according to claim 6, further comprising adding a glucide with respect to which the microorganism has a metabolic ability.

8. The method for producing imidazole dipeptide according to claim 1, wherein the microorganism is a peptidase-deficient strain.

9. The method for producing imidazole dipeptide according to claim 8, wherein the peptidase is PepD.

10. A method for producing anserine in which the imidazole dipeptide is carnosine, comprising further reacting carnosine produced by the microorganism according to claim 2 with an enzyme having carnosine N-methyltransferase activity.

11. The method for producing anserine according to claim 10, wherein a microorganism expressing an enzyme having carnosine N-methyltransferase activity is used.

12. The method for producing anserine according to claim 10, wherein a microorganism coexpressing an enzyme having carnosine synthesizing activity and an enzyme having carnosine N-methyltransferase activity is used as the L-amino acid α-ligase.

13. The method for producing anserine according to claim 10, wherein the enzyme having the carnosine N-methyltransferase activity is represented by SEQ ID NO: 36.

14. The method for producing anserine according to claim 10, wherein the production method is a fermentation method.

15. The method for producing imidazole dipeptide according to claim 2, wherein the microorganism is a microorganism expressing L-amino acid α-ligase.

16. The method for producing imidazole dipeptide according to claim 4, wherein
the L-amino acid α-ligase,
is a mutant L-amino acid α-ligase in which are substituted 1 to 3 amino-acid residues of a wild-type YwfE amino-acid sequence that has an amino-acid sequence represented by SEQ ID NO: 12, and includes
a substitution of an amino-acid residue corresponding to the 108th asparagine (N) residue from the N-terminus of SEQ ID NO: 12 with an amino acid selected from alanine (A), glutamic acid (E), and glutamine (Q),
a substitution of an amino-acid residue corresponding to the 112th isoleucine (I) residue from the N-terminus of SEQ ID NO: 12 with valine (V),
or
a substitution of an amino-acid residue corresponding to the 378th histidine (H) residue from the N-terminus of SEQ ID NO: 12 with an amino acid selected from lysine (K) or arginine (R).

17. The method for producing imidazole dipeptide according to claim 2, wherein the production method includes a bacterial reaction method or a fermentation method.

18. The method for producing imidazole dipeptide according to claim 3, wherein the production method includes a bacterial reaction method or a fermentation method.

19. The method for producing imidazole dipeptide according to claim 4, wherein the production method includes a bacterial reaction method or a fermentation method.

20. The method for producing imidazole dipeptide according to claim 5, wherein the production method includes a bacterial reaction method or a fermentation method.
